(19) United States
(12) Patent Application Publication
(10) Pub. No.: US 2002/0049404 A1
(43) Pub. Date: Apr. 25, 2002

(54) FAS LIGAND EXPRESSING ANTIGEN PRESENTING CELLS FOR TOLERANCE INDUCTION

(76) Inventors: JOHN D. MOUNTZ, BIRMINGHAM, AL (US); TONG ZHOU, BIRMINGHAM, AL (US)

Correspondence Address: BENJAMIN A. ADLER, 8011 CANDLE LANE, HOUSTON, TX, 77071, US

(21) Appl. No.: 09/079,834

(22) Filed: May 15, 1998

Related U.S. Application Data
(60) Provisional application No. 60/046,560, filed on May 15, 1997.

(51) Int. Cl. A61M 1/00; A01N 63/00; A01N 65/00; A61K 35/26; A61K 35/28

(52) U.S. Cl. 604/28; 424/577; 424/578

(57) ABSTRACT

The present invention provides a method of inducing systemic tolerance to an antigen in an individual in need of such treatment, comprising the step of: administering antigen presenting cells to said individual, wherein said cells express Fas ligand and said antigen.

### FEDERAL FUNDING LEGEND

This invention was produced in part using funds obtained through grants AR44982, NO1-AR-62224, P50AI23649 and R01-AR42547 from the National Institutes of Health. Consequently, the federal government has certain rights in this invention.

### BACKGROUND OF THE INVENTION

1. Field of the Invention

The present invention relates generally to immunology. More specifically, the present invention relates to the use of Fas ligand technology for tolerance induction in the treatment of autoimmune disease, graft rejections and in the use of gene therapy.

2. Description of the Related Art

Antigen presenting cells (APCs) play important roles in the initiation of the T-cell response and the induction of T-cell tolerance (Bretscher & Cohn, 1970; Lafferty & Gill, 1993; Jenkins & Schwartz, 1987). Induction of complete T-cell activation and proliferation requires the provision of two signals by the antigen 10 presenting cells. In the absence of the second signal, the so called costimulatory signal, the T cells become anergic (Liu & Linsley, 1992; June et al., 1990; 1994; Linsley & Ledbetter, 1993). The ability of antigen presenting cells to guide the CD4-positive T-cell response, skewing it toward either a predominantly Thl or Th2 response also influences the development of many autoimmune diseases (Sayegh et al., 1995; Corry et al., 1994; Lu et al., 1994; Guerder et al., 1994a, 1994b; Harlan et al., 1994). The different antigen presenting function of the antigen presenting cells determines the profile of the T-cell response. Thus, antigen presenting cells can determine whether an immune response is immunogenic or tolerogenic (Finkelman et al., 1996).

Antigen presenting cells also influence the response to allografts (Lafferty et al., 1983). It has long been recognized that the graft rejection reaction is most intense in tissues that contain lymphoreticular elements, but is relatively less intense in other tissues, such as muscle. The ability of graft tissue to stimulate a rejection response is diminished greatly by the removal of antigen presenting cells from the graft tissue prior to transplantation (Lafferty et al., 1976; La Rosa & Talmage, 1983). The presence of viable antigen presenting cells in the graft is crucial to initiation of the rejection response (Steinman et al., 1993). Both purified MHC class I and class II alloantigens elicit only a weak response unless directly presented by viable donor antigen presenting cells (Warrwns et al., 1994). Decreased expression of either MHC antigens or costimulatory molecules, such as B7, on donor antigen presenting cells greatly increases survival of allogeneic grafts (Lenschow et al., 1992; Turka et al., 1992). Thus, direct antigen presentation by donor antigen presenting cells plays a critical role in initiation of graft rejection by induction of a strong T cell response.

The importance of Fas-mediated apoptosis in the maintenance of T-cell tolerance and prevention of autoimmune disease has been demonstrated by the finding that mutations of the Fas or Fas ligand genes leads to autoimmune disease in lpr/lpr and gld/gld mice, respectively (Watanabe-Fukunaga et al., 1992; Suda et al., 1993). Clonal deletion of peripheral T cells after antigen stimulation is defective in Fas-deficient lpr/lpr mice (Singer & Abbas, 1994). The maintenance of T-cell tolerance to self-antigen and superantigen is defective in Fas-deficient lpr/lpr mice (Zhou et al., 1991, 1992, 1994). Furthermore, correction of the Fas-mediated apoptosis defect in T cells by expression of a fas transgene prevents autoimmune disease in lpr/lpr mice and an age-related defect in Tcell apoptosis in aged mice (Wu et al., 1994; Zhou et al., 1995). Fas is expressed on the cell surface and mediates apoptosis when ligated by Fas ligand or agonistic anti-Fas antibody (Itoh et al., 1991; Yonehara et al., 1989; Suda et al., 1993). The induction of activation-induced cell death (AICD) occurs through an autocrine response involving Fas and Fas ligand expressed by the individual T cells (Ju et al., 1995; Brunner et al., 1995; Dhein et al., 1995), indicating the significance of Fas-mediated apoptosis in the maintenance of T-cell tolerance.

Fas-mediated apoptosis of antigen presenting cells contributes to down-modulation of the immune response. Activated T cells express elevated levels of Fas ligand and induce apoptosis of antigen presenting cells (Ashany et al., 1995). On the other hand, activated macrophages express Fas ligand and are able to induce apoptosis of the T cells (Oyaizu et al., 1997). For example, the high level of expression of Fas ligand by HIV-infected macrophages has been implicated in the depletion of CD4-positive T cells in AIDS (Dadley et al., 1996). Fas ligand expression on dendritic cells may play a critical role in regulation of T cell response (Schular et al., 1997; Lu et al., 1997).

Fas-mediated apoptosis also plays a role in the maintenance of immunoprivileged sites. The immunoprivileged status of the testis and anterior chamber of the eye requires a high level of expression of Fas ligand in the parenchymal cells of these organs (Griffith et al., 1995). In this situation, it has been suggested that the expression of Fas ligand by the parenchymal cells protects these tissues from destruction by T cells through induction of apoptosis of the T cells. As inoculation of virus into the anterior chamber of the eye leads to systemic T-cell tolerance to the virus, the immune-privileged status of this site may involve induction of systemic T-cell tolerance in addition to induction of local T-cell tolerance (Griffith et al., 1996). It has been proposed that the antigen presenting cells expressing Fas ligand together with the privileged antigen that are released from the immune-privileged sites mediate apoptosis of the peripheral T cells, thus inducing systemic T-cell tolerance. Transplantation of allogeneic or xenogeneic tissues that do not express Fas ligand into the testis o r the anterior chamber of the eye prevented rejection to these tissues (Streilein, 1993; Head et al., 1983; Benson & Niederkorn,1992; Maddocks & Setchell, 1990). Direct evidence for the role of Fas ligand in prevention of graft-rejection has been provided by the finding that implantation of syngeneic muscle cells that express Fas ligand around allogeneic grafted-islets leads to long-term acceptance of the transplanted islets as well as local induction of T-cell apoptosis by the Fas ligand expressing cells around the graft (Lau et al., 1996). Maintenance of tolerance to the graft required the presence of expression of Fas ligand by the syngeneic muscle cells. This finding suggests a practical approach to the prevention of graft rejection in transplantation through manipulation of Fas ligand-mediated apoptosis. The function of Fas ligand in the grafts has been questioned, however, and it may not confer prolonged survival but induce an inflammatory response (Kang et al., 1997; Biancone et al., 1997; Allison et al., 1997). Thus, the mechanisms underlying immune-privilege have not been fully elucidated.

Adenovirus gene therapy is limited by the induction of an immune response to the virus or the gene-therapy protein product (Yang & Wilson, 1995; Christ et al., 1997; Yang et al., 1995; Gilgenkrantz et al., 1995). A specific T-cell response to the adenovirus results in the failure to re-administer the gene therapy (Yang et al., 1994; Juillard et al., 1995). Previous attempts to reduce the T-cell response to the adenovirus during gene therapy, including blockade of MHC class I and II antigens, reduction in the antigenicity of the adenovirus, and prevention of co-stimulation of T cells, either have not fully eliminated the response or invoke general immunosuppression (Yang & Wilson, 1995; Christ et al., 1997; Yang e t al. 1995; Gilgenkrantz et al., 1995; Yang et al., 1994; Juillard et al., 1995; Yang et al. 1996; Schowalter et al., 1997; Qin et al., 1997; Guerette et al., 1996; Zsengeller et al. 1997).

An ideal strategy for elimination of the immune response would be induction of peripheral T-cell tolerance that is specific for the adenoviral vector. Clonal deletion of antigen-specific T cells, which is mediated by apoptosis, is an important mechanism in the maintenance of peripheral T-cell tolerance (Bellgrau et al., 1995; French et al., 1996; Lee et al., 1997; Griffith et al., 1996). Activation-induced cell death in T cells, in which apoptosis of the T cells is mediated by upregulation of Fas and Fas ligand, also contributes to down-regulation of the T cell response (Suda et al., 1995; Watanable-Fukunaga et al., 1992; Zhou et al., 1992; Suda et al. 1993; Wu et al. 1994; Cheng et al. 1997).

Recently, it has been shown that Fas ligand can create immune-privileged sites and prevent graft rejection by inducing apoptosis in T cells entering the site (Lau et al., 1995; Griffith et al., 1995; Muruve et al., 1997; Muruve et al., 1997; Sigalla et al., 1997; DMatteo et al., 1995). T cell tolerance induction has been shown to prolong adenovirus expression (Hamilton et al., 1997; Zepeda & Wilson, 1996; Ilan et al., 1997; Bennett et al., 1996). Whether introduction of antigen presenting cells expressing high levels of Fas ligand together with a specific antigen might induce specific, systemic tolerance to the antigen is unknown.

Thus, the prior art is deficient in new and potent mechanisms of immunological tolerance induction involving Fas ligand technology. The present invention fulfills this long-standing need and desire in the art.

### SUMMARY OF THE INVENTION

The present invention demonstrates that antigen presenting cells that express Fas ligand and processed adenovirus antigens can directly induce apoptosis of Fas-positive T cells resulting in adenovirus-specific T-cell tolerance. High levels of Fas ligand and adenovirus antigens were induced in antigen presenting cells by co-infection with AdLoxpFasL and AxCANCre. Pre-treatment of recipient mice with the adenovirus-infected antigen presenting cells that express Fas ligand resulted in induction of T-cell tolerance to the adenovirus, and prolonged the expression of LacZ transgene after administration of AdCMVLacZ. Thus, pre-tolerization with syngeneic antigen presenting cells co-infected with AdLoxpFasL and AxCANCre may be a novel immunointervention strategy for tolerance induction to adenovirus gene therapy.

The present invention demonstrates that treatment with allogeneic antigen presenting cells that express the Fas ligand induces a profound alloantigen-specific T-cell unresponsiveness. Using H-2D^{b}/H-Y TCR transgenic mice, the present invention shows that this rapid and profound depletion of antigen-specific T cells contributes to the induction of systemic T-cell tolerance. These results indicate that Fas ligand-expressing antigen presenting cells can induce T-cell tolerance that is both systemic and antigen-specific. This form of induction of T-cell tolerance requires expression of Fas on the T cells, as well as Fas ligand expression on the antigen presenting cells.

The current results demonstrate that AdLoxpFasL coinfection with AxCANCre results in very high levels of Fas ligand expression in almost 100% of infected antigen presenting cells. One reason for this high efficiency of infection is that both viruses can be grown to very high titers in the 293 cells as there is no Fas ligand expression by AdLoxpFasL, which requires co-infection with a AxCANCre virus (Zhang et al., 1998). Second, the two-virus system was used to infect an APC cell line derived from Fas mutant C57BL/6-lpr/lpr mice. Therefore, these antigen presenting cells can express high levels of Fas ligand without undergoing autocrine suicide. Third, there is very high efficiency of infection of these antigen presenting cells with adenovirus as disclosed in the present invention. This is in contrast to low efficiency transfection of DNA into antigen presenting cells using lipofectin (1%-5%) or electroporation (8%). Therefore, the present technique utilizes several unique technologies to allow high expression of Fas ligand together with high expression of processed adenovirus antigen on a n APC that can induce apoptosis of T cells that react with this antigen.

In an embodiment of the present invention, there is provided method of inducing systemic tolerance to an antigen in an individual in need of such treatment, comprising the step of: administering antigen presenting cells to said individual, wherein said cells express Fas ligand and said antigen.

In another embodiment of the present invention, there is provided a method of inducing T-cell tolerance to a virus in an individual receiving gene therapy, comprising the steps of: transfecting Fas ligand-expressing antigen presenting cells with said virus; introducing said transfected antigen presenting cells into said individual; and treating said individual with said virus for the purpose of gene therapy, wherein said antigen presenting cells expressing the Fas ligand induce T-cell tolerance to said virus.

In yet another embodiment of the present invention, there is provided a method of increasing expression of a transgene in an individual, comprising the steps of: administering antigen presenting cells to said individual, wherein said cells express Fas ligand and an antigen to the protein product of said transgene; and delivering to said individual a viral vector encoding a transgene wherein said antigen presenting cells induce apoptosis of Fas-positive T-cells resulting in an increased expression of the transgene.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 shows the phenotypic analysis of a macrophage cell line for use as antigen presenting cells. The peritoneal resident macrophages from C57BL/6-lpr/lpr mice were isolated and cultured. The macrophages were tested for expression of Fas

(FIG. 1A), Mac-1 (FIG. 1B), F4180 (FIG. 1C), IA^{b} (FIG. 1D), H-2D^{b} (FIG. 1E), and B7 (FIG. 1F) by flow cytometric analysis. 10,000 viable cells were analyzed by FACScan. The open histograms are controls for isotype antibody staining.

FIG. 2 shows the characterization of Fas ligand expressing macrophages. The macrophages were transfected with a pcDNAIII expression vector (Invitrogen) containing a full length murine Fas ligand cDNA, or empty vector, using electroporation. Transfected macrophages were selected with 0.5 mg/ml G418 (Sigma).

FIG. 2A). The Fas ligand activity of the selected macrophages was determined by mixing macrophages with ⁵¹Cr-labeled A20 cells at the indicated ratios and after an 8 hour incubation, the specific release was determined.

(FIG. 2 B). Splenic T cells were purified from 4-week-old MRL/MpJ-+/+ and MRL/MpJ-lpr/lpr mice (Jackson Laboratory) using a T-cell enrichment column (R&D Systems). Purified T cells (5×10⁵) were cultured with γ-irradiated macrophages (5×10⁴) in round-bottom, 96-well plates for 5 days, and proliferation was determined b y adding 1 μCi of ³H-thymidine (Amersham), 16 h prior to harvest.

FIG. 3 shows the T cell proliferative response of treated MRL-+/+and lpr/lpr mice to H-2^{b} alloantigen. Four-week-old MRL-+/+

(FIG. 3A) and −Ipr/lpr

(FIG. 3B) mice were injected intraperitoneally with 2×10⁶ macrophages transfected with Fas ligand or control vector every 3 days for 6 times. On day 3 of the final injection, the splenic T cells were isolated from treated mice and cultured with 2×10⁵ γ-irradiated total spleen cells from C57BL/6 +/+ mice. T-cell proliferation was determined by incorporation of ³H-thymidine at indicated time points. The error bars indicate the mean ±SEM for 5 mice analyzed separately in triplicate assays.

FIG. 4 shows T cell proliferative response of treated MRL-+/+ (FIG. 4A) or −Ipr/lpr (FIG. 4B) mice to H-2^{d} alloantigen and CD3 crosslinking. Four-week-old MRL-+/+ mice were injected intraperitoneally with 2×10⁶ macrophages transfected with Fas ligand or control vector every 3 days for 6 times. On day 3 of the final injection, the splenic T cells were isolated from treated mice and cultured with 2×10⁵ γ-irradiated total spleen cells from BALB/c +/+ mice, or 5 μg/ml anti-CD3 antibody. T-cell proliferation was determined by incorporation of ³H-thymidine at indicated time points. The error bars indicate the mean +SEM for 5 mice analyzed separately in triplicate assays.

FIG. 5 shows high levels of Fas ligand expression in H2D^{b} H-Y macrophages transduced by the recombinant adenoviruses. Peritoneal macrophages were prepared and transfected with the recombinant adenoviruses containing Fas ligand cDNA.

(FIG. 5A). Flow cytometry analysis of Fas ligand expression: 1×10⁶ cells were stained with anti-murine Fas ligand antibody and analyzed b y FACScan. Solid histogram indicates Fas ligand positive cells.

(FIG. 5B). ⁵¹Cr release assay for Fas ligand activity. Transfected macrophages were co-cultured with 15Cr labeled A20 cells at indicated ratios for 8 hours, and Fas ligand activity was determined by the specific release of 51Cr from A20 target cells.

FIG. 6 shows the T cell proliferative response of treated TCR transgenic B6-+/+(Tg-+/+) (FIG. 6A) or Tg-lpr/lpr (FIG. 6B) mice to macrophages expressing the Fas ligand and the H-2D^{b}/H-Y antigen (closed circles) or control macrophages (open circles).

FIG. 7 shows the T cell response to H-2D^{b} H-Y antigen in treated female TCR transgenic-+/+ and lpr/lpr mice. Tolerance induction was carried out in female, TCR transgenic D^{b}/HY-+/+ or lpr/lpr mice using CD4⁻CD8⁻ T cells from female, C57BL/6-lpr/lpr mice and male C57BL16-lpr/lpr mice with or without co-administration of the Fas-Ig fusion protein. (FIG. 7A) Expression of M33, CD8, and Fas on the T cells in the peripheral lymph node cells was determined by three-color flow cytometry analysis. 10⁶ total peripheral lymph node cells were stained with biotin-conjugated M33, and then with FITC-conjugated anti-CD8 and PE-conjugated anti-Fas (PharMingen). 10,000 viable lymphocytes were analyzed by FACScan. Two-color contour plots of CD8 and M33 are shown, and the percentage of M33⁺CD8⁺ T cells is indicated in the upper right quadrant. (FIG. 7B) Kinetic analysis of the M33⁺CD8⁺ T cells in the spleen. At each indicated time point, total spleen cells were stained and analyzed as described above. The absolute number of M33⁺CD8⁺ T cells was calculated by the percentage of the M33⁺CD8⁺ T cells multiplied by the total number of spleen cells. The error bars indicate the mean ±SEM for 3 mice analyzed. (FIG. 7C) Fas expression on M33⁺CD8⁺ T cells. The M33⁺CD8⁺ T cells were gated as shown in FIG. 4A, and the percentage of Fas expression on the gated M33⁺CD8⁺ T cells is indicated.

FIG. 8 shows the detection of Fas ligand expressing antigen presenting cells and the induction of apoptosis in vivo. Spleen cells (FIGS. 8A-8D) and liver cells (FIGS. 8E, 8F) were examined by H&E staining (FIGS. 8A, 8B) or in situ TUNEL staining (FIGS. 8C, 8D) following systemic administration of Fas ligand-expressing H-2D^{b}/H-Y macrophages.

FIG. 9 shows that co-infection of antigen presenting cells with AdloxpFasL+AxCanCre (APC−AdFasL) results in high levels of expression of Fas ligand that are capable of inducing apoptosis of A20 target cells. The AdLoxpFasL was infected into antigen presenting cells from B6-lpr/lpr mice with and without AxCANGe. As a comparison, antigen presenting cells were transfected b y electroporation with pcDNA3FasL or stimulated with lipopolysaccharide (LPS) (1 μg/ml). FasL expression was determined by the ability of the transfected antigen presenting cells to induce apoptosis of a ⁵¹Cr labeled, Fas- sensitive cell line A20.

FIG. 10 shows the prolongation of transgene expression by Ad/FasL expressing antigen presenting cells. Ten-week-old B6-+/+ mice were treated with 1×10⁶ of the antigen presenting cells co-infected with AdLoxpFasL plus AxCANCre (APC-AdFasL) or antigen presenting cells co-infected with AdLoxpFasL plus AdCMVLuc (APC-AdControl) every 3 days for 5 doses. After 7 days, mice were inoculated intravenously with 1×10¹⁰ pfu AdCMVLacZ. At the indicated time points, LacZ gene expression in the liver was analyzed by a quantitative assay (FIG. 10A) and in situ LacZ histochemical staining (FIG. 10B). LacZ histochemical staining indicates A; APC-AdControl Day7, B; AdControl Day30, C; APC-AdFasL Day 7, D; APC-AdFasL Day 30. The error bars indicate the mean ±SEM for 3 mice analyzed separately in triplicate assay

FIG. 11 shows the inhibition of CD3⁺ T cell expansion in the spleen after cell therapy with antigen presenting cells coinfected with AdLoxpFasL+AxCANCre. B6-+/+ mice were treated with 1×10⁶ of either APC only (left), APC-AdControl (middle), or APC-AdFasL (right) every 3 days for 5 doses. Seven days later, all treated mice were challenged with AdCMVLacZ (1×10¹⁰ pfu). Seven days after the second challenge, mice were sacrificed and the spleen was analyzed for CD3⁺ T cells by immunohistochemical staining. These results are representative of immunohistochemical stains of 5 mice/group. (×320)

FIG. 12 shows the induction of tolerance to 20 adenovirus by APC-AdFasL. Ten-week-old B6-+/+ mice were injected intravenously with 1×10⁶ APC-AdFasL or APC-AdControl every 3 days for 5 doses as described above. On day 7 after the final injection, mice were challenged with AdCMVlacZ and the T-cell cytotoxic response against APC plus adenovirus was determined by killing of APC cells infected with AdCMVGFP (5 pfu/cell). The percentages of viable GFP expressing APC cells were quantitated by FACS analysis. Background cytotoxicity of APC cells infected with AdCMVGFP was less than 10% at all times points. The error bars indicate the mean ±SEM for 3 mice analyzed separately in triplicate assays.

FIG. 13 shows decreased IFN-γ and IL-2 induction by tolerized B6-+/+ mice. B6-+/+ (FIGS. 13A, 13B) and B6-lpr/lpr (FIGS. 13C, 13D) mice were treated with APC-AdFasL or APCAdControl cells (1×10⁶). On day 7 after the final injection, mice were challenged with AdCMVlacZ. Seven days later, splenic T-cell were isolated from the mice and incubated for 24 h with irradiated antigen presenting cells that were either uninfected or infected with adenovirus. Levels of IL-2 (FIGS. 13A, 13C) and IFN-γ (FIGS. 13B, 13D) in the supernatant was determined by ELISA.

FIG. 14 shows that Ad/FasL antigen presenting cells induce specific T-cell tolerance to adenovirus. C57BL/6-+/+ mice (5 mice/group) were treated with either APC-AdFasL or APC-AdControl. Seven days later, mice were challenged in vivo with either AdCMVLacZ or mouse cytomegalovirus (MCMV). After an additional 7 days, splenic T-cells were stimulated in vitro with antigen presenting cells alone, or antigen presenting cells infected with MCMV or AdCMVLacZ. IL-2 production in the supernatants was determined by ELISA 48 h later.

### DETAILED DESCRIPTION OF THE INVENTION

Autocrine interaction of Fas and Fas ligand leads to apoptosis of activated T cells, a process that is critical for maintenance of peripheral T-cell tolerance. Paracrine interactions of Fas ligand with T cells also may play an important role in the maintenance of tolerance as Fas ligand can create immune-privileged sites and prevent graft rejection by inducing apoptosis in the T cells. It was surmised that antigen presenting cells that express Fas ligand might directly induce apoptosis of T cells during presentation of antigen to the T cells, thus inducing antigen-specific, systemic T-cell tolerance. The present invention demonstrates that profound, specific T-cell unresponsiveness to alloantigen was induced by treatment of H-2^{k} mice with H-2^{b} antigen presenting cells that expressed Fas ligand, and that profound T-cell unresponsiveness specific for the H-Y antigen was induced by treatment of H-2D^{b}/H-Y TCR transgenic female mice with H-2D^{b}/H-Y antigen presenting cells that expressed the Fas ligand. The induction of this systemic T-cell tolerance required the expression of Fas ligand on the antigen presenting cells as well as expression of Fas on the T cells. The tolerance was restricted to the antigen presented by the antigen presenting cells. The rapid and profound clonal deletion of the antigen-specific, peripheral T cells mediated by the Fas ligand expressing antigen presenting cells contributed to the induction of tolerance. The present invention demonstrates that antigen-specific T-cell tolerance can be induced by antigen presenting cells that express Fas ligand and suggest a novel function for antigen presenting cells in the induction of T-cell apoptosis. Furthermore, they indicate a novel immunointervention strategy for treatment of graft rejection and autoantigen-specific autoimmune diseases.

Furthermore, the present invention demonstrates that the immune response to adenovirus was prevented by induction of specific T-cell tolerance by pre-treatment with adenovirus-infected antigen-presenting cells that express Fas ligand. A major problem associated with adenovirus gene therapy is the T cell-mediated immune response, which is elicited by inoculation of the adenovirus vector and leads to rapid clearance of the virus and loss of transgene expression. Compared to control-treated mice, the tolerized mice showed prolonged expression of LacZ upon administration of AdCMVLacZ 1 week after tolerance induction. In contrast to the control treated mice, the tolerized mice did not display in vivo proliferation of CD3+ T cells in the spleen in response to AdCMVLacZ, and splenic T cells exhibited lower production of interferon-y and interleukin-2 in response to AdCMVLacZ infected antigen presenting cells in vitro. The T-cell tolerance was specific for the adenovirus, as the T-cell responses to mouse cytomegalovirus (MCMV) remained unimpaired. Adenovirus-specific T-cell tolerance can be induced by antigen presenting cells that co-express the Fas ligand and adenovirus antigens. The invention disclosed herein provides a new strategy that can be used to induce tolerance to adenovirus vector gene therapy with resultant prolonged expression of the transgene.

The present invention is directed towards a method of producing antigen-specific T-cell tolerance to inhibit or eliminate graft rejection, autoimmune dieseases and the immune response developed in response to adenovirus during gene therapy. This is achieved by treatment with antigen presenting cells expressing the Fas ligand and a specific antigen.

The present invention is directed towards a method of inducing systemic tolerance to an antigen in an individual in need of such treatment, comprising the step of: administering antigen presenting cells to said individual, wherein said cells express Fas ligand and said antigen. The antigen presenting cells induce apoptosis of Fas-positive T-cells directed towards said antigen, thereby resulting in said induction of specific, systemic tolerance to said antigen. Representative examples of antigen include the adenovirus antigen, an autoantigen, a viral antigen, an adenoassociated viral antigen, and an alloantigen. In one embodiment, the individual has an autoimmune disease. Representative examples of autoimmune diseases include diabetes, multiple sclerosis, rheumatoid arthritis, thyroiditis, Grave's disease, systemic lupus erythematosus. In another embodiment, the individual has had an organ transplant. In another embodiment, the individual has a decreased cytotoxic T cells and decreased CD4 helper cells. Importantly, the various methods of the present invention disclosed herein may further comprise the step of delivering to said antigen presenting cells a gene to inhibit apoptosis. A representative example of a gene to inhibit apoptosis is crmA.

The present invention is directed towards a method of inducing T-cell tolerance to a virus in an individual receiving gene therapy, comprising the steps of: transfecting Fas ligand-expressing antigen presenting cells with said virus; introducing said transfected antigen presenting cells into said individual; and treating said individual with said virus for the purpose of gene therapy, wherein said antigen presenting cells expressing the Fas ligand induce T-cell tolerance to said virus.

The present invention is also directed towards a method of increasing expression of a transgene in an individual, comprising the steps of: administering antigen presenting cells to said individual, wherein said cells express Fas ligand and an antigen to the protein product of said transgene; and delivering to said individual a viral vector encoding a transgene wherein said antigen presenting cells induce apoptosis of Fas-positive T-cells resulting in an increased expression of the transgene.

The present invention is also directed towards a method of creating immune-privileged sites in an individual so as to decrease rejection of a graft, comprising the steps of: extracting antigen presenting cells from donor organ tissue; introducing Fas ligand into said antigen presenting cells to produce Fas ligand-expressing antigen presenting cells expressing an antigen specific to said graft; introducing said Fas ligand-expressing antigen presenting cells expressing an antigen specific to said graft to said individual prior to and during said grafting procedure; wherein said Fas ligand-expressing antigen presenting cells expressing an antigen specific to said graft create create said immune-privileged site at the site of said grafting procedure to prevent rejection of said graft in said individual. In another embodiment, there is provided a method decreasing rejection of a graft in an individual, comprising the steps of: perfusing donor organ tissue with Fas ligand; introducing said donor organ tissue to said individual.

The following examples are given for the purpose of illustrating various embodiments of the invention and are not meant to limit the present invention in any fashion:

### EXAMPLE 1

Animals

MRL-+/+ mice, MRL-lpr/lpr mice, C5BL/6-+/+ and Ipr/lpr mice were originally purchased from the Jackson Laboratory (Bar Harbor, Mass.) and bred in the UAB animal facility. C57BL/6 H2D^{b}/H-Y reactive TCR transgenic +/+ and lpr/lpr mice were generated as described (Osmound et al., 1994).

### EXAMPLE 2

Construction of Fas Ligand Expression Adenovirus Vector

Construction of Fas ligand expression adenovirus vector was carried out as described (Zhang et al., 1998). Briefly a 10.4 kb shuttle vector containing the fragment of adenovirus from 0 map unit to 1 map unit followed by the 1.6 kb chicken β-actin promoter plus CMV enhancer. This was followed by 2 Loxp sites separated by a Neo resistant gene plus a 0.3 kb bovine growth hormone poly A tail. The full-length 0.9 kb FasL was cloned down-stream from the bovine growth hormone poly A tail which was followed by an SV40 polyA tail and by the 9.8-16.1 map units of adenovirus.

### EXAMPLE 3

Generation of Macrophage Cell Line and Transfection for Fas Ligand Expression

Male or female C57BL/6-lpr/lpr of 8 to 12 weeks-of-age were injected intraperitoneally with 1 ml of pristane to facilitate production of macrophages. The peritoneal macrophages were prepared and the isolated macrophages were transfected with a recombinant adenoviruses expression system.

Macrophages were isolated from the peritoneal cavity of C57BL/6-lpr/lpr mice. Isolated macrophages were stimulated with lipopolysaccharide (100 ng/ml) for 24 hours every 10 days. After three cycles, the macrophages were grown in 10% FCS-RPMI 1640 in preparation for transfection. Macrophages (5×10⁶/ml) were electroporated with purified pcDNAIII plasmid (10 μg) containing full-length murine Fas ligand cDNA at 960 mF, 250 mV using a gene pulser (BioRad). Transfected cells were cultured in 10-cm culture dishes for 48 hours and selected with 0.5 mg/ml G418 for 4 weeks.

Murine B6-lpr/lpr antigen presenting cells were infected with either AdLoxpFasL plus AxCANCre (APC-AdFasL) o r AdLoxpFasL plus AdCMVLUC (APC-AdControl) at 5 pfu/cell of each virus for 1 h at 37° C., and the infected cells incubated at 37° C. for a further 24 h (Zhang et al. 1998). Expression of murine Fas ligand and adenoviral antigens on the surface of B6-lpr/lpr antigen presenting cells was identified using an indirect immune fluorescent assay (Sigalla et al., 1997) and the functional ability of Fas ligand in mediating killing was evaluated using a ⁵¹Cr-release assay as described (Zhang et al. 1998).

### EXAMPLE 4

Phenotypic Analysis by Flow Cytometric Analysis

Anti-CD3 (clone: 145.2Cl 1), anti-CD4 (clone: GK 1.5), anti-CD8 (clone: 53-47), anti-Mac-1 (clone: 1M/70), anti-Fas (clone: Jo2), anti-IA^{b} (clone: AF6-120.1), anti-H-2D^{b} (clone: 28-14-8) were purchased from Pharmingen (San Diego, Calif.). The anti-D^{b}/H-Y TCR clonotypic mAb M33 was produced as described previously (22). Single cell suspensions of thymocytes or lymphnode cells were labeled with optimal concentrations of FITC-conjugated anti-CD8, PE-conjugated anti-CD4, or PE-conjugated anti-Fas (Pharmingen, San Diego, Calif.) and biotin-conjugated M33 followed by Tandem-Streptavidin. Viable cells (10,000/sample) were analyzed by flow cytometry on a FACS-Scan (Becton Dickinson, Mountain View, Calif.) equipped with logarithmic scales and the data processed in a Hewlett-Packard (Palo Alto, Calif.) computer. The number of cells in each population was determined by quadrant analysis of contour graphs. 10,000 viable cells were analyzed by FACScan.

### EXAMPLE 5

⁵¹Cr Release Assay for Fas Ligand Activity

A murine B lymphoma cell line (A20), which is very sensitive to Fas ligand-induced cytotoxicity, was used as the target cells. A20 cells (5×10⁶/ml) were incubated with 0.3 m Ci ⁵¹Cr sodium in complete medium (1 ml) for 45 min. After thorough washing, labeled A20 cells (1×10⁵) were incubated with effector cells at different effector to target (E/T) ratios starting at 10:1 in 200 ml of complete medium for 12 hours. The same number of labeled A20 cells were culture in 200 ml of complete medium alone to determine spontaneous release and in complete medium with 0.01% SDS to determine maximum release. 100 ml of supernatant was collected and counted. Specific release was calculated as follows: Specific release (%)=(CPM of sample−CPM of minimum release)/(CPM of maximum release−CPM of minimum release).

Fas ligand expression was determined by the ability of the AdLoxpFasL+AxCanCre transfected antigen presenting cells to induce apoptosis of a ⁵¹Cr labeled, Fas-sensitive cell line, A20 (Zhang et al., 1998). Target cells (1×10⁶), which are sensitive to cytotoxic lysis, were incubated with 20 μCi of [⁵¹Cr]-sodium chromate in 100 μl of RPMI-1640 containing 10% FCS at 37° C. for 1 h. After washing with medium, these cells were used as target cells. Effector cells were prepared from B6-lpr/lpr antigen presenting cells infected with AdLoxpFasL plus AxCANCre as described. These effector cells were then incubated with [⁵¹Cr]-labeled target cells (1×10⁴) at different effector/target (E/T) ratios in a total volume of 200 μl of the medium. Release of ⁵¹Cr into the supernatant was assessed 6 h later using a β-counter.

### EXAMPLE 6

Adenovirus-specific Cytotoxic T Cell Activity Using AdCMVGFP-infected Target Cells

The adenovirus shuttle vector construct was produced b y cloning the enhanced GFP gene (Clonetech) into the HindIII-XbaI site of pCA13 (Microbix, Canada). This was cotransfected with pJM17 to produce recombinant AdCMVGFP. AdCMVGFP was plaque purified by 3 rounds of selection. These were used to infect APC to be used as target cells for analysis of cytotoxic effector T cells from mice treated with APC-AdFasL and APC-AdControl. Effector T cells were prepared from spleen of AdCMVLacZ-immunized and nonimmunized mice. These effector cells were then incubated with AdCMVGFP-infected target cells (1×10⁵) at different effector/target (E/T) ratios in round-bottom microtiter plates in a total volume of 200 μl of the medium for 48 h, and Green fluorescent-positive APC were sorted using FACS analysis.

### EXAMPLE 7

Induction of Allogeneic T-cell Tolerance by Fas Ligand Expressing Macrophages

Female MRL-+/+ and lpr/lpr mice 4 to 6 weeks old were injected intravenously with 5×10⁶ macrophages transfected with Fas ligand expression vector or macrophages with empty vector as a control. The injection was repeated every three days for six times. Three days after the last injection, mice were sacrificed for evaluation of tolerance induction.

Ten-week-old C57BL/6-+/+ mice were injected intravenously with 1×10⁶ of the antigen presenting cells co-infected with AdLoxpFasL plus AxCANCre (APC-AdFasL) or antigen presenting cells co-infected with AdLoxpFasL plus AdCMVLUC (APC-AdControl) or with PBS every 3 days for 5 doses. On day 7 after the final injection, mice were challenged with AdCMVlacZ and the T cell cytotoxic response against APC infected with adenovirus was determined 1 week after challenge.

Female, H-2D^{b}/HYTCR transgenic +/+ and lpr/lpr mice of 4 to 6 weeks-of-age were injected intravenously once with 5×10⁶ adenoviruses-transfected macrophages as described. T-cell tolerance was analyzed at day 1 through day 21 after the last injection.

### EXAMPLE 8

Analysis of the T-cell Proliferative Response

To determine the allogeneic T-cell response, T cells were purified from the spleen and peripheral lymph node of treated MRL-+/+ and lpr/lpr mice using a T-cell enrichment column. Purified T cells (5×10⁵) were cultured in 96-well, round-bottom plates in a total volume of 200 ml with medium alone, 5 mg/ml anti-CD3, 5 mg/ml concanavilin-A or an equal number of γ-irradiated (3300 rad) allogeneic spleen cells from CS7BL/6 (H-2^{b}) or BALB/c (H-₂^{d}) mice. For analysis of the H-2D^{b}/H-Y-specific T-cell response, T cells were purified from treated TCR transgenic mice as described, cultured in the presence of 50 U/ml of murine interleukin-2 (IL-2) (Genzyme, Cambridge Mass.) for the indicated time with irradiated (3300 rad) syngeneic spleen cells obtained from C57BL/6 male or female mice. At the indicated time points, 1 μCi of ³H-thymidine was added, the cells were harvested 16 h later, and incorporation of ³H-thymidine was determined using a scintillation counter (Wallace).

### EXAMPLE 9

Analysis of T-cell Mediated Cytotoxicity

Peripheral T cells purified from the spleen using a murine T-cell enrichment column (R&D Systems, Inc.) were stimulated with irradiated spleen cells obtained from C57BL/6 male mice in the presence of 50 U/ml of IL-2 for 3 days. Viable cells were collected by centrifugation over Ficoll. Con A stimulated spleen cells from C57BL/6 male or female mice were labeled with ⁵¹Cr and mixed with stimulated T cells at the indicated ratio. After incubation for 8 h, the ⁵¹Cr released in the supernatants was measured using a γ-counter (Packard Bell, St. Louis, Mo.) and the specific release calculated using a standard method.

### EXAMPLE 10

Analysis of the Immune Response to Adenovirus and MCMV After Tolerance Induction

One week after tolerance induction, mice were treated with AdCMVlacZ (1×10¹⁰ pfu i.v.) or MCMV (1×10⁵ pfu i.v.). After an additional 7 days, purified splenic T-cells were stimulated in vitro with antigen presenting cells alone, or antigen presenting cells that have been incubated either with MCMV or AdCMVlacZ. After 48 h the supernate was collected and analyzed for IL-2 expression.

### EXAMPLE 11

Ouantitation of β-galactosidase Expression in Liver

Freshly isolated liver tissue was homogenization for 10 s in a tissumizer in 1 ml of β-gal buffer (Tropix, Inc., Bedford Mass.) (Young et al., 1993). The homogenate was centrifuged at 12,500× g for 10 min at 4° C., and the supernatant was heated for 60 min at 48° C. to inactivate the endogenous eukaryotic β-galactosidase activity. β-galactosidase activity was determined using the Galacto-light™ (Tropix, Inc., Bedford Mass.) chemi-luminescent reporter assay. The protein concentration was determined by the Bradford assay (BioRad). The activity is expressed as the relative light units/min/mg of total protein in the liver.

### EXAMPLE 12

Cytokine Production in Vitro in Response to Antigen Presenting Cells Infected with Adenovirus

B6-lpr/lpr antigen presenting cells were infected with AdCMVLacZ (10 pfu/cell) for 1 hour in 1 ml of media and then diluted by addition of 10 ml of RPMI1640 supplemented with 10% fetal bovine serum and culture continued at 37° C. for 24 hours. Before use as target cells, the antigen presenting cells were γ-irradiated, and 1×10⁵ antigen presenting cells were mixed with different ratios of T cells isolated from the spleen of tolerized mice. The mixed cells were incubated in 96-well plates for 2 d at 37° C. The supernatants were collected and induction of IL-2 and IFN-γ w as determined using an ELISA assay kit (R & D systems Inc., Minn.).

Tissues were then embedded in paraffin blocks, sectioned (10 μm thickness) and stained with hematoxylin and eosin. Anti-CD3 (Dako Corporation, Carpinteria, Calif.) was detected by the avidin-biotin conjugate (ABC) immunohistochemical technique.

### EXAMPLE 13

Characterization of Antigen Presenting Cells that Express Fas Ligand

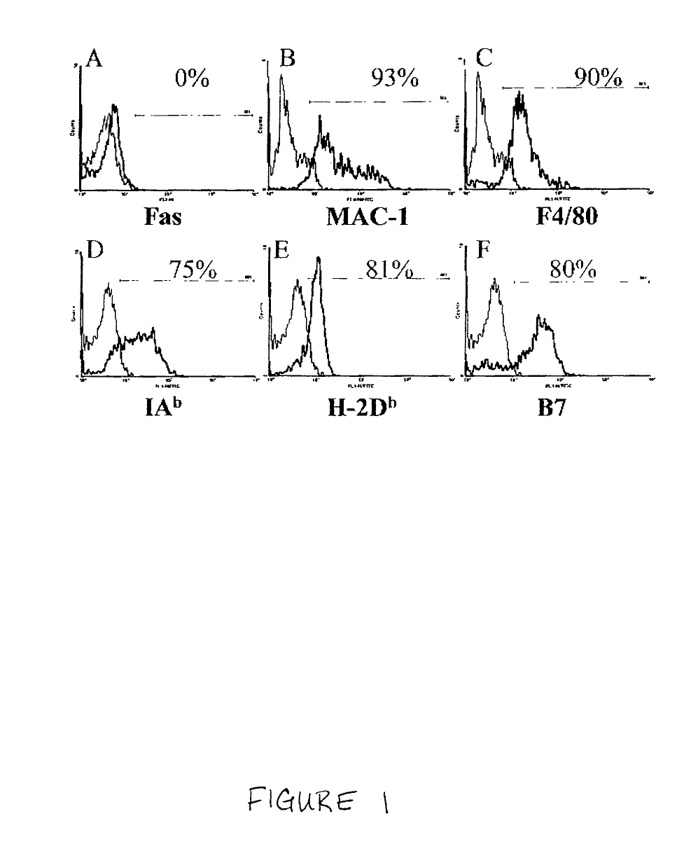

The peritoneal macrophages were used as antigen presenting cells. Since most peritoneal macrophages express Fas and are susceptible to Fas ligand-induced apoptosis after activation, a non-transformed peritoneal macrophage cell line from fas mutant C57BL/6-lpr/lpr mice was derived. As expected, these macrophages did not express Fas (FIG. 1A), but did express most of the typical phenotypic markers of macrophages, including Mac-1 (FIG. 1B), and F4/80 (FIG. 1C). The macrophages expressed high levels of MHC class II IA^{b} antigens (FIG. 1D), intermediate levels of MHC class I H-2D^{b} antigens (FIG. 1E), and significant levels of the B7 costimulatory molecule, as detected by a CTLA4-Ig fusion protein (FIG. 1F). As this long-term cultured cell line retains the characteristic phenotype of macrophages, this macrophage cell line was used as a source of antigen presenting cells.

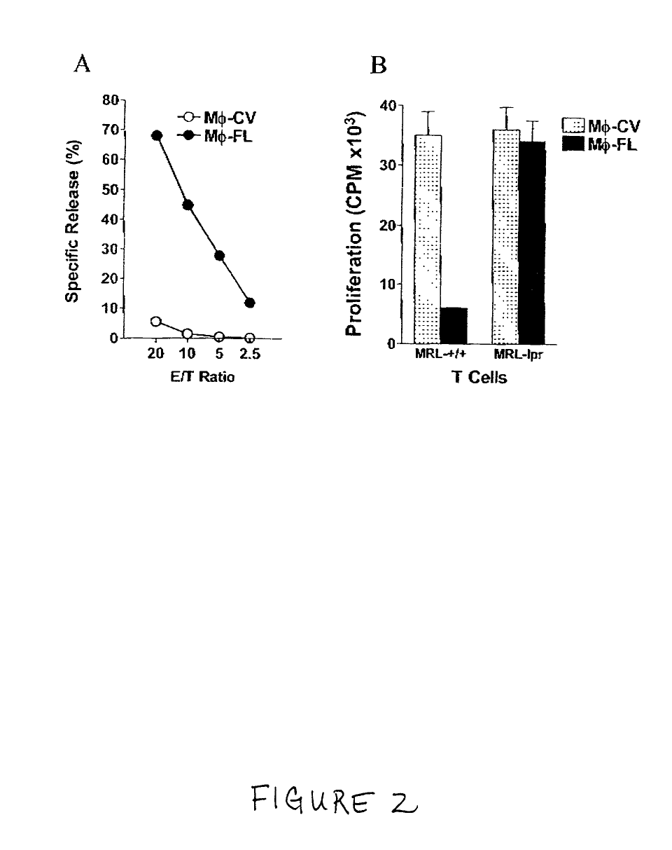

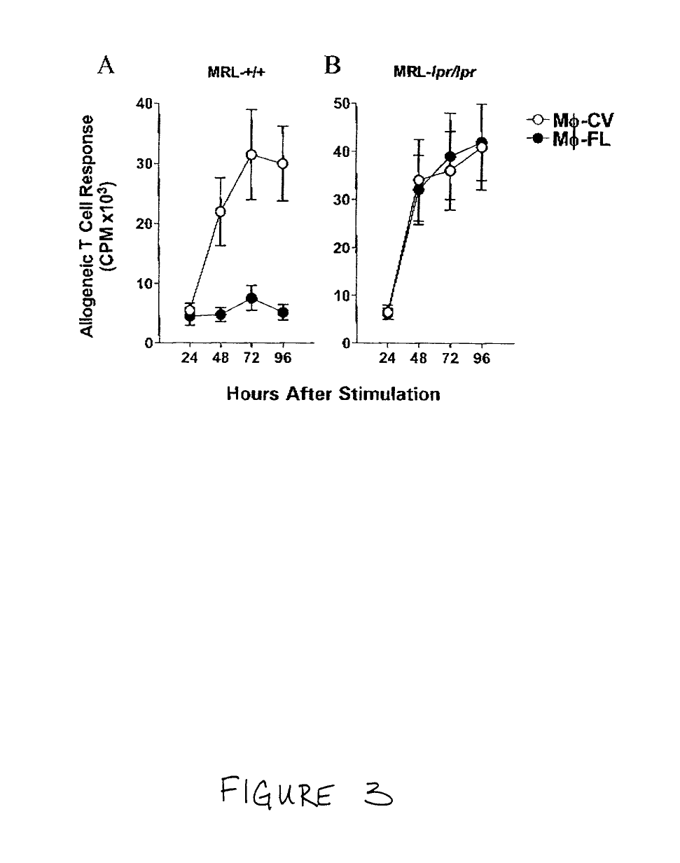

To generate antigen presenting cells that express the Fas ligand, the macrophage cell line was transfected with a eukaryotic expression vector (pcDNAIII) containing a full-length murine Fas ligand cDNA, using transfection with the empty vector as a control and selection with G418. The macrophages transfected with Fas ligand (Mφ-FL), but not those transfected with control vector (Mφ-CV), exhibited high levels of Fas ligand activity as shown by the specific release of ⁵¹Cr from Fas ligand-sensitive A20 target cells (FIG. 2A).

To further determine the effect of expression of Fas ligand of macrophages on the allogeneic T cells response in vitro, purified T cells from an MHC-mismatched mouse strain, MRL (H-2^{k}), were co-cultured with γ-irradiated Mφ-CV or Mφ-FL. The Mφ-CV cells induced proliferative responses in T cells from either MRL-+/+ or MRL-lpr/lpr mice (FIG. 2B), indicating that this cell line is capable of presenting alloantigen and inducing allogeneic T cell response. In contrast, Mφ-FL cells did not induce a proliferative response in T cells obtained from MRL-+/+mice, indicating that Fas ligand expressing cells inhibit allogeneic T-cell response. The presence of Mφ-FL cells did not alter the allogeneic T-cell response of MRL-lpr/lpr mice, which do not express functional Fas, indicating that the inhibition of allogeneic T cell response by Fas ligand expressing macrophages requires Fas expression on the T cells, and is specific for Fasmediated apoptosis.

### EXAMPLE 14

Induction of T-cell Unresponsiveness by Allogeneic Antigen Presenting Cells that Express Fas Ligand

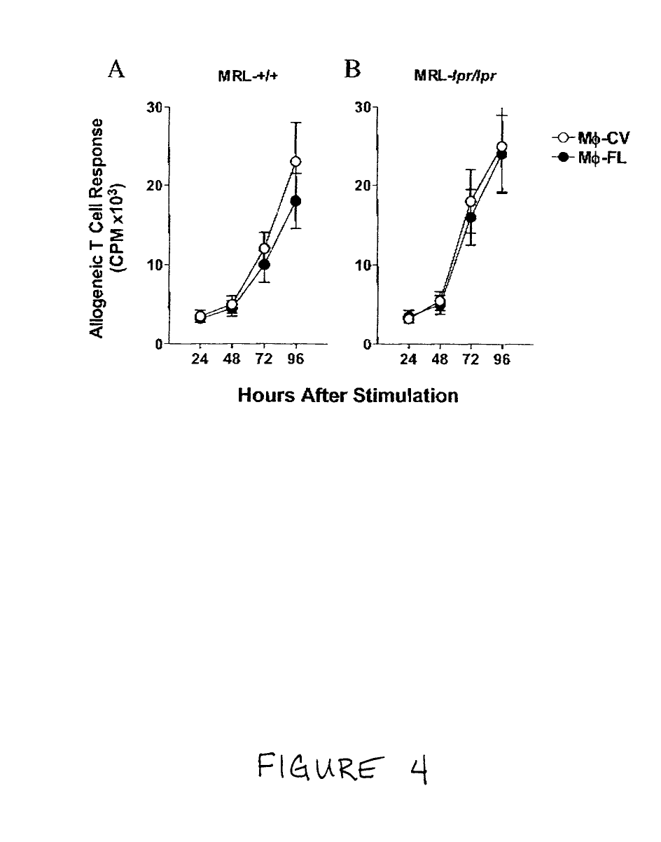

To determine whether antigen presenting cells that express Fas ligand can induce alloantigen-specific T-cell tolerance in vivo, Mφ-FL or Mφ-CV cells (H-2^{b}) was administered through six intra-peritoneal injections given at 3-day intervals to 4-week-old MRL-+/+ and MRL-lpr/lpr (H-2k) mice. Three days after the last injection, splenic T cells from treated MRL mice were co-cultured with γ-irradiated total spleen cells from C57BL/6 (H-2b) mice. Treatment of MRL-+/+ mice with H-2^{b} macrophages that express the Fas ligand considerably reduced the proliferative T-cell response to H-₂^{b} alloantigen during a 96-hour culture, whereas treatment with control macrophages had no effect (FIG. 3A). This result indicates that treatment with macrophages that express the Fas ligand induces T-cell unresponsiveness to the alloantigen. To determine whether expression of Fas is required for induction of T-cell tolerance, MRL-lpr/lpr mice were treated similarly. In these mice, the T-cell response to the H-2^{b} antigen was not affected by treatment with Fas ligand expressing macrophages (FIG. 3B), indicating that Fas expression is required for induction of T-cell unresponsiveness.

### EXAMPLE 15

T-cell Unresponsiveness is Specific for the Alloantigen presented

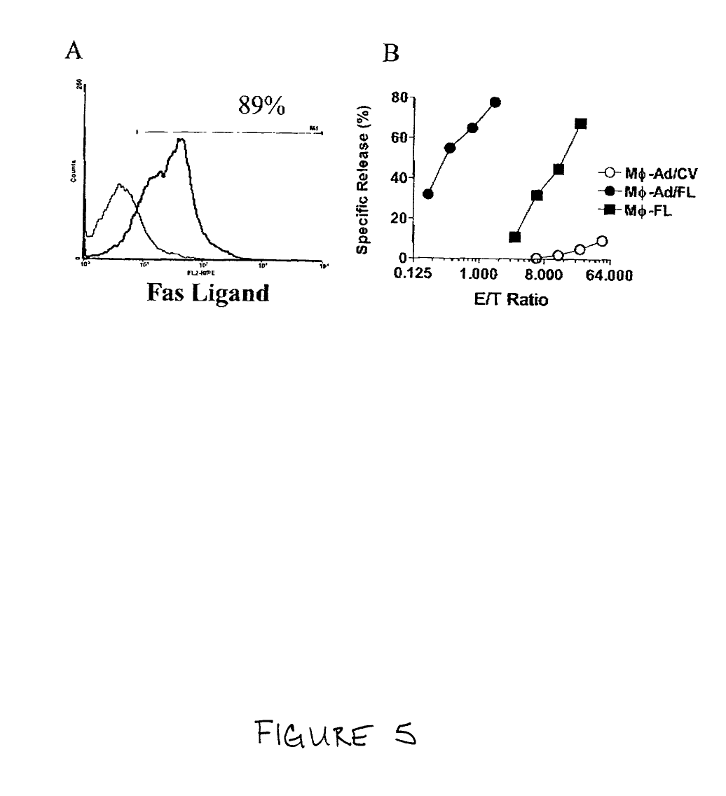

To determine whether the T-cell unresponsiveness induced by Fas ligand expressing macrophages is alloantigen specific, T cells from MRL-+/+ mice treated with Fas ligand expressing H-2^{b} macrophages were analyzed for their proliferative response to a control alloantigen, H-2^{d}, expressed on cells from BALB/c mice. The T cell response to H-2^{d} in both MRL-+/+ and MRL-lpr/lpr mice was unaffected by treatment with either Fas ligand expressing H-2^{b} or control macrophages (FIGS. 4A and B). These results indicate that the induced T-cell unresponsiveness is specific for the alloantigens borne on the Fas ligand-expressing macrophages. The T cell proliferative response to crosslinking with anti-CD3 antibody was found to be similar for T cells obtained from MRL-+/+ mice treated with Fas ligand-expressing macrophages or control macrophages, indicating that the treatment with Fas ligand expressing macrophages does not result in non-specific immunosuppression.

### EXAMPLE 16

Induction of H-2D^{b}/H-Y-specific T-cell Tolerance by Fas Ligand Expressing Antigen Presenting Cells in H-2D^{b}/H-Y TCR Transgenic Mice

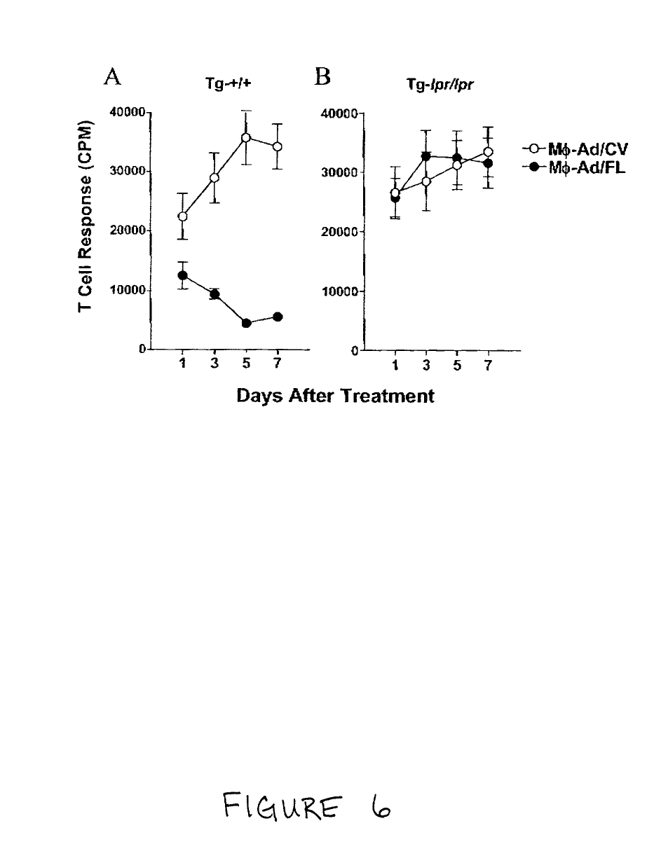

In order to determine the mechanisms by which Fas ligand expressing antigen presenting cells induce systemic and antigen-specific T-cell tolerance, T-cell receptor (TCR) transgenic, H-2D^{b}/H-Y-reactive mice were used. In the female transgenic mice, the majority of peripheral (D8⁺ T cells bear the transgenic TCR and are reactive with the male H-Yantigen presented in the context of the H-2D^{b} antigen (Kisielow et al., 1988). Peritoneal macrophages isolated from male CS7BL/6-lpr/lpr mice were used as the antigen presenting cells. Because conventional transfection technique were unable to induce high levels of Fas ligand expression on the primary macrophages, the recombinant adenoviruses that contain Fas ligand cDNA were used (Zhang et al., 1998). High levels of Fas ligand expression in these freshly isolated macrophages were obtained. Flow cytometric analysis showed that nearly 90%of the macrophages that were transfected with the Fas ligand adenoviruses expressed high levels of the Fas ligand in comparison with those transfected with control viruses (FIG. 5A). The activity of the Fas ligand was determined by a ⁵¹Cr release assay (FIG. 5B), the macrophages transfected with Fas ligand adenoviruses (MΦ-Ad/FL; closed circles) exhibited the highest Fas ligand activity compared to those obtained using a conventional method (MΦ-FL; closed squares), and those transfected with the control viruses (MΦ-Ad/CV; open circles). Thus, high Fas ligand-expressing and H-2D^{b}/HY antigen presenting cells were generated using Fas ligand adenoviruses transfected macrophages obtained from C57BL/6 male mice.

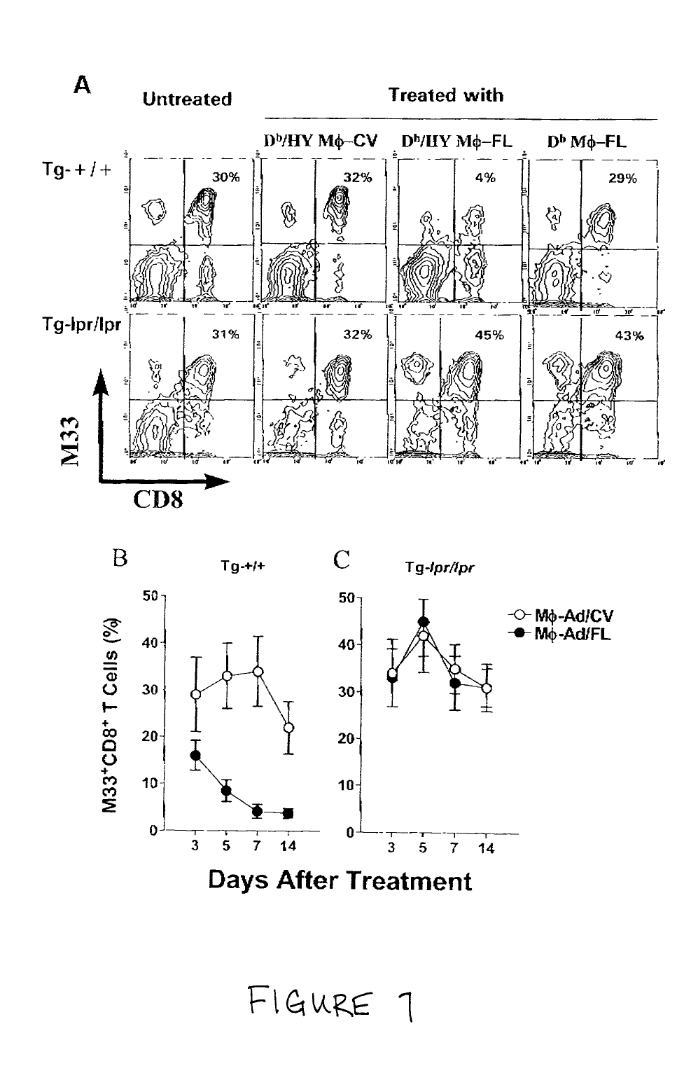

H-2D^{b}/H-Y T-cell receptor transgenic female mice were injected intra-peritoneally with the Fas ligand transfected macrophages that expressed H-2B^{b}/H-Y antigen and the T-cell response to H-2D^{b}/H-Y antigen was kinetically analyzed. TCR transgenic female B6-+/+ (Tg-+/+) mice received macrophages expressing Fas ligand and the H-2D^{b}/H-Y antigen (Mφ-Ad/FL) exhibited greatly decreased T-cell proliferative response to the H2D^{b}/H-Y antigen (FIG. 6A; closed circles). Inhibition of T cells response to the H-2B^{b}/HY antigen was observed as early as day 1, and persisted at a low level at day 7 after treatment. In contrast, the transgenic female mice received control macrophages (Mφ-Ad/CV) exhibited a graduately increased T cell response to the H-2D^{b}/HY antigen, presumably due to pre-stimulatory effect of macrophage treatment. This result indicates that Fas ligand expression on H-2D^{b}/H-Y bearing macrophages is capable of inducing T-cell unresponsiveness to the H-Y antigen while the control macrophages prime the response. As described for the induction of alloantigen unresponsiveness, the expression of Fas antigen on the responding T-cells is required as treatment of TCR transgenic female lpr/lpr mice with Fas ligand-positive, H-2D^{b}/H-Y expressing macrophages did not affect the T-cell proliferative response (FIG. 6B). Similar treatment with either Fas ligand positive or negative macrophages from female mice, which express H-2D^{b}, but not the HY antigen, did not lead to a significant decrease in the T-cell response, indicating that H-Y antigen contexed with H-2D^{b} is also required for induction of T cell tolerance.

### EXAMPLE 17

Rapid and Profound Clonal Deletion of H-2D^{b}/H-Y Reactive T Cells Induced by Fas Ligand Expressing Antigen Presenting Cells

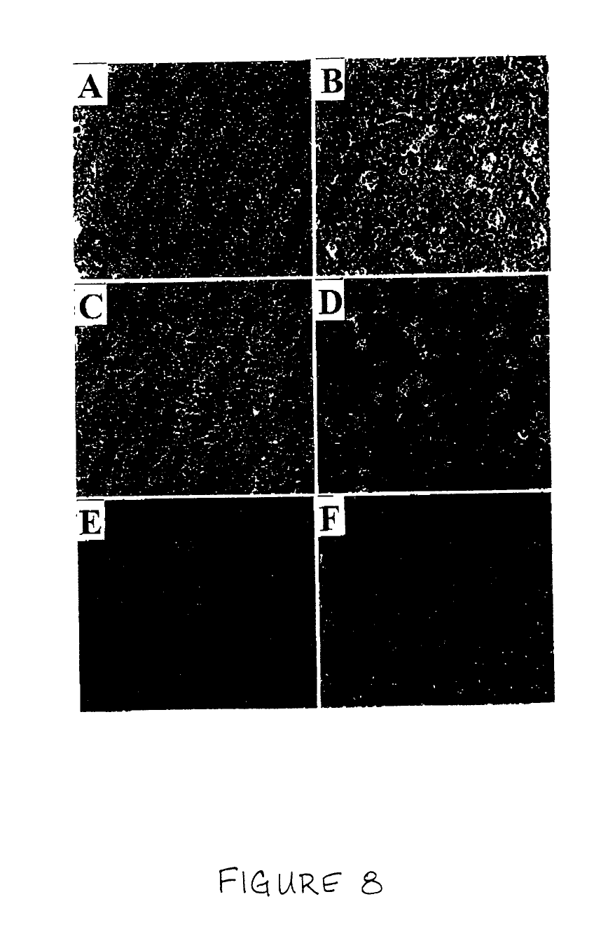

The ability of antigen presenting cells that express Fas-ligand to induce clonal deletion of antigen-specific T cells was tested directly in female, TCR transgenic mice. The use of these TCR transgenic mice allowed examination of the clonal deletion of the H2D^{b}/H-Y specific T cells by analyzing the numbers of M33⁺ CD8⁺ T cells in female, TCR transgenic mice (Kisielow et al., 1988). Tolerance induction was carried out and the numbers of H-2D^{b}/H-Y-specific T cells in the spleen were determined by staining with an anti-TCR clontypic antibody (M33) and CD8 seven days after treatment. In both untreated TCR transgenic B6-+/+ and B6-lpr/lpr mice, approximately 30% of the splenic T cells were M33+CD8+ (FIG. 7A). Treatment of TCR transgenic female B6-+/+ or B6-lpr/lpr mice with Fas ligand-negative, H-2D^{b}/H-Y macrophages (D^{b}/Hφ-CV) obtained from male mice, did not affect the numbers of M33⁺CD8⁺ T cells. This result indicates that H-2D^{b}/H-Y antigen alone does not lead to a significant reduction in the number of antigen-specific T cells seven days after treatment. In contrast, the numbers of M33+CD8⁺ T cells were significantly reduced in TCR transgenic +/+ mice (less than 5%) treated with H-2D^{b}/H-Y macrophages that expressed Fas ligand (D^{b}/HY Mφ-FL). As similar treatment of TCR transgenic -lpr/lpr mice did not affect the numbers of M33+CD8⁺ T cells (38%), these results indicate that the Fas ligand expressing antigen presenting cells induce clonal deletion of antigen-specific T cells through Fas/Fas ligand-mediated apoptosis. In addition, the HY antigen was also required for induction of clonal deletion, because the transgenic female mice treated with Fas ligand-expressing macrophages without the HYantigen did not exhibit reduced number of M33+CD8⁺ T cells.

Since the H-2D^{b}/H-Y antigen alone could cause specific T-cell clonal deletion through activation-induced T-cell suicide, which is also mediated by Fas and Fas ligand, the time course and efficiency of T-cell deletion mediated by Fas ligand expressing antigen presenting cells was examined and compared to that mediated by antigen presenting cells alone. Kinetic analysis of M33^{+CD}8⁺ T cells showed that depletion of M33⁺CD8⁺ T cells in TCR transgenic B6-+/+ female mice occurred as early as day 3 after treatment with H-2D^{b}/H-Y cells expressing Fas ligand, as less than 20% of the T cells were M33⁺ CD8⁺ (FIG. 7B). In contrast, in TCR transgenic B6-+/+ mice treated with H-2D^{b}/H-Y cells that did not express the Fas ligand, the number of M33⁺CD⁺ T cells underwent a gradual decrease after day 7 of treatment, but was never below 20%. Treatment of transgenic-lpr/lpr mice with either Fas ligand-positive or -negative H-2D^{b}/H-Y macrophages did not cause significant deletion of splenic M33⁺CD8⁺ T cells at any time point, but rather a slight increase in the numbers of M33⁺CD8⁺ T cells after treatment with either Fas ligand-positive H-2D^{b}/H-Y macrophages was observed at day 5. Although there was a decrease in the numbers of M33⁺CD8⁺ T cells at day 14 and day 21 in treated transgenic-lpr/lpr mice, the depletion was much less than that observed in TCRtransgenic +/+ mice treated with Fas ligand-negative, H-2D^{b}/H-Y cells (FIG. 7C), indicating that the AICD of activated T cells is defective in lpr/lpr mice. Female H-2D^{b} cells did not affect the numbers of M33⁺CD8⁺ T cells in any group of mice, neither were M33⁻CD8⁺ T cells affected by any of the treatments in either TC transgenic +/+ or lpr/lpr mice.

In summary, these results indicate that the Fas ligand-expressing H-2D^{b}/H-Y, but not the Fas ligand-negative cells, induce an early occurring and more efficient deletion of M33⁺CD8⁺ T cells; a process that is Fas-dependent as it occurred in TCR transgenic B6-+/+ but not B6-lpr/lpr mice. Furthermore, the depletion of M33⁺CD8⁺ T cells in TCR transgenic B6-+/+ mice requires the presence of the H2D^{b}/H-Y antigen as H-2D^{b} cells from female mice, which lack the H-Y antigen, did not induce deletion. Moreover, the deletion is antigen specific because M33⁺CD8⁺ but not M33-CD8+ T cells were deleted, and lastly, the Fas ligand-expressing antigen presenting cells induced deletion differs from Fas-mediated activation-induced cell death in both timing and intensity. Fas ligand-expressing cells induce an early occurring and more complete clonal deletion of the responding T cells, whereas activation-induced cell death occurs at a later time point and is incomplete.

### EXAMPLE 18

Fas Ligand Expressing Macrophages Primarily Migrated into Lymphoid Organs and Induced Apoptosis of T Cells

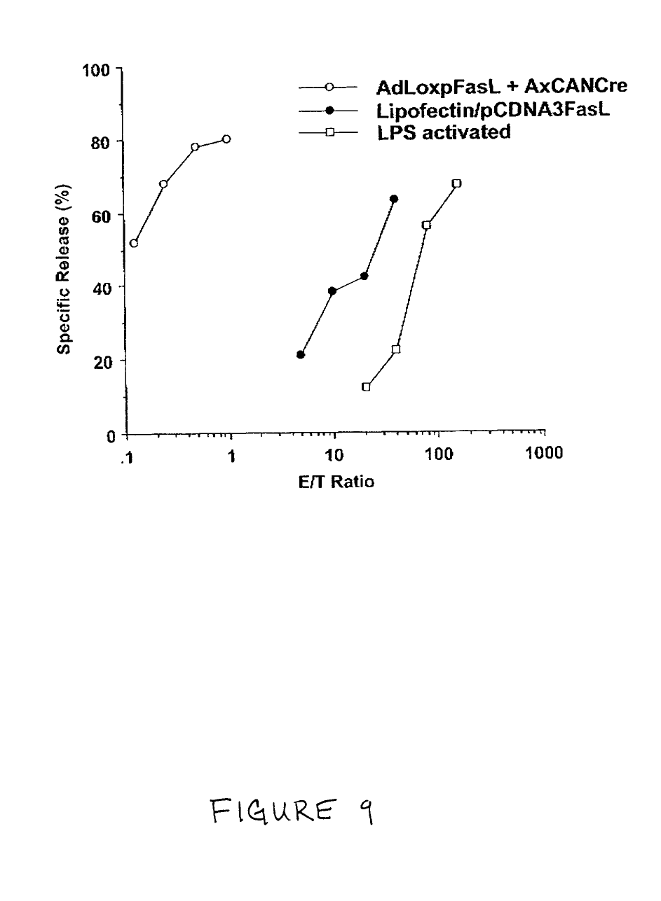

It has been reported that local expression of high levels of Fas ligand results in neutrophil infiltration and tissue inflammation. To rule out the possibility that systemic administration of Fas ligand expressing macrophages causes inflammation, the migration of Fas ligand expressing macrophages after systemic administration was examined. Both Fas ligand transfected and control macrophages migrated into the spleen equivalently after intra-peritoneal injection. 48 hours after injection, the spleen from H-2D^{b}/HY TCR transgenic female mice that received Fas ligand expressing H-2D^{b}/HY macrophages did not exhibit a significant inflammatory response compared to that in the mice that received control macrophages as demonstrated by H&E staining (FIGS. 8A, B). However, there was increased number of apoptotic cells in the spleen of the transgenic mice that received Fas ligand expressing macrophages but not those that received control macrophages as demonstrated by in situ TUNEL staining (FIGS. 8C, D). Apoptotic cells in the spleen were clustered, presumably due to the killing by Fas ligand-expressing macrophages of the T cells. Apoptosis induced by Fas ligand-expressing macrophages was also specific for Fas, because there was no apoptosis in the spleen of lpr/lpr mice. Because systemic administration of soluble Fas ligand or anti-Fas antibody causes severe liver damage, the liver of B6-+/+ mice that received Fas ligand-expressing macrophages were also examined. No significant damage of the liver was observed (FIGS. 8E, F). These results indicate that Fas ligand-expressing macrophages primarily migrate into and reside in the spleen, and do not cause inflammatory response in the spleen and liver damage. Thus, using macrophages as carriers to deliver Fas ligand is a safe strategy for Fas ligand based therapy.

### EXAMPLE 19

Co-infection of AdLoXpFasL and AxCANCre Results in High Levels of Fas Ligand Expression which Induces Apoptosis of A20 Target Cells

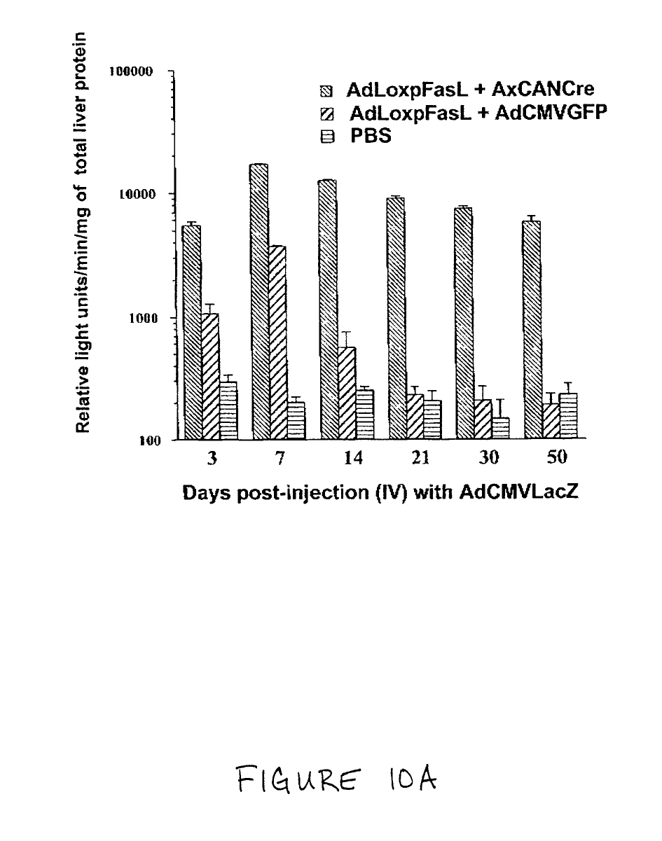

A novel AdLoxpFasL-modified adenovirus was developed to allow high titer production of the virus in 293 cells (Zhang et al. 1998). This technique also allows control of Fas ligand expression since FasL is not expressed in the absence of co-infection with AxCANCre. This strategy was used to induce high levels of Fas ligand expression in an antigen presenting cell line derived from Fas-mutant B6-lpr/lpr mice. The lytic activity of the antigen presenting cells infected with AdLoxpFasL+AxCANGe (APC−AdFasL) against A20 target cells was approximately 100-fold higher than that of antigen presenting cells transfected by lipofectin with a pcDNA3-FasL expression vector, or stimulated with lipopolysaccharide (LPS)-activated (FIG. 9A). The expression of high levels of Fas ligand by the antigen presenting cells was sustained for at least 7 days of in vitro culture (FIG. 9B).

### EXAMPLE 20

Pretreatment with Antigen Presenting Cells/AdFasL Therapy Prolonged AdCMVLacZ-induced Expression of LacZ in the Liver

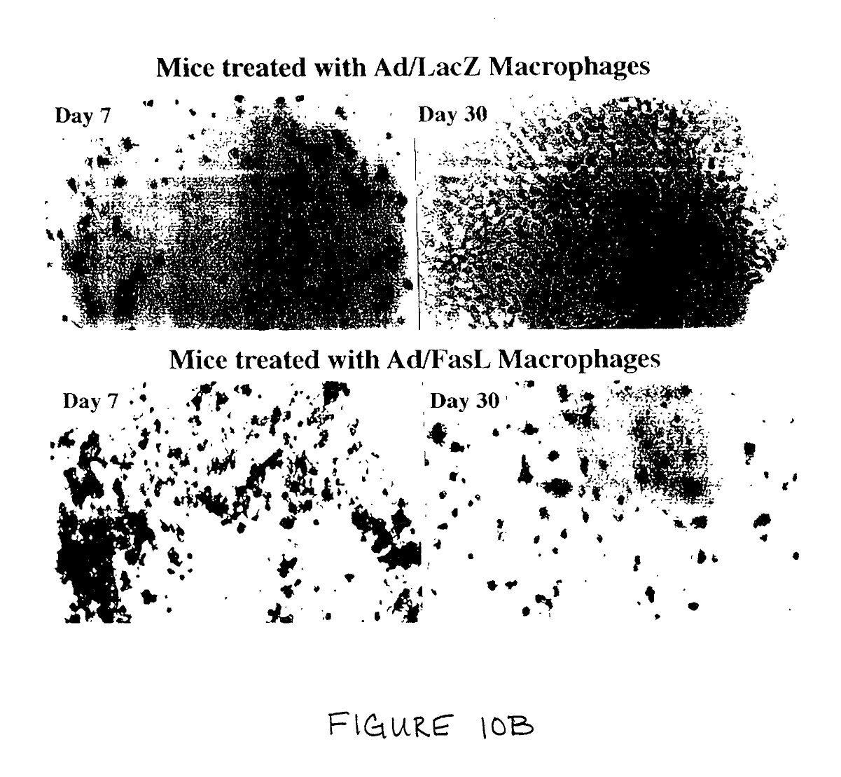

Expression of adenovirus gene therapy in the liver is limited due to an acute inflammatory response and a chronic cytotoxic T-cell response (Yang & Wilson, 1995; Christ et al., 1997; Yang et al., 1995; Gilgenkrantz et al., 1995; Yang et al., 1994; Juillard et al., 1995; Yang et al., 1996; Schowalter et al., 1997; Qin et al., 1997; Guerette et al., 1996; Zsengeller et al., 1997). To determine if treatment with APC-AdFasL leads to prolongation of LacZ expression delivered by adenoviral vector, the APC-AdFasL-treated and APCAdControl-treated mice were inoculated with AdCMVlacZ (1×10¹⁰ pfu). The levels of LacZ gene expression in the liver decreased rapidly in mice treated with antigen presenting cells infected with AdLoxpFasL+AdCMVLuc (APC−AdControl) (FIG. 10A). In contrast, in mice treated with APC-AdFasL, the levels of LacZ gene expression were sustained for at least 50 days after gene delivery (FIG. 10A). At day 30 after delivery of AdCMVlacZ, LacZ positive cells were detectable by immunohistochemical analysis in the liver of mice that had been pretreated with Ad/FasL expressing antigen presenting cells (FIG. 10B, lower panel), whereas there were few LacZ-positive cells in the liver of the mice that were pretreated with APC-AdControl (FIG. 10B, upper panel). These results indicate that pretreatment with APC-AdFasL significantly prolongs AdCMVlacZ transgene expression.

### EXAMPLE 21

Decreased T-cell Expansion in APC-AdFasL-treated Mice

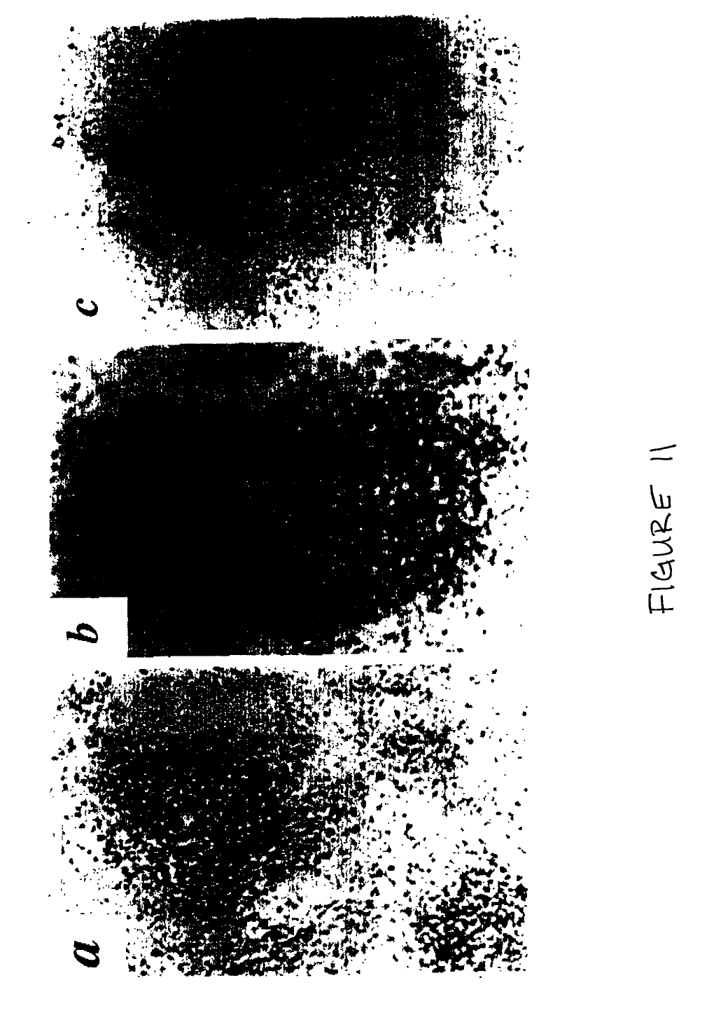

B6+/+ mice were treated with APC-AdFasL or APCAdControl every 3 days for 5 doses, and then all treated mice were challenged intravenously with AdCMVIacZ (10×10¹⁰ pfu). Three days later, the spleen from naive mice (FIG. 11A), APC-AdControl treated mice (FIG. 11B), and APC-FasL treated mice (FIG. 11C) were stained with anti-CD3 antibody. Expansion of the CD3⁺60 T cell population was not observed in the spleens of APC-AdFasL tolerized mice, whereas clonal expansion of CD3⁺ T cells was observed in the spleens of mice treated with APC-AdControl after challenge. These results suggest that APC-AdFasL induces tolerance in treated mice.

### EXAMPLE 22

Decreased Cytotoxic Response of T Cells to AdCMVGFP-infected Target Cells after Tolerance with Fas Ligand Expressing Antigen Presenting Cells

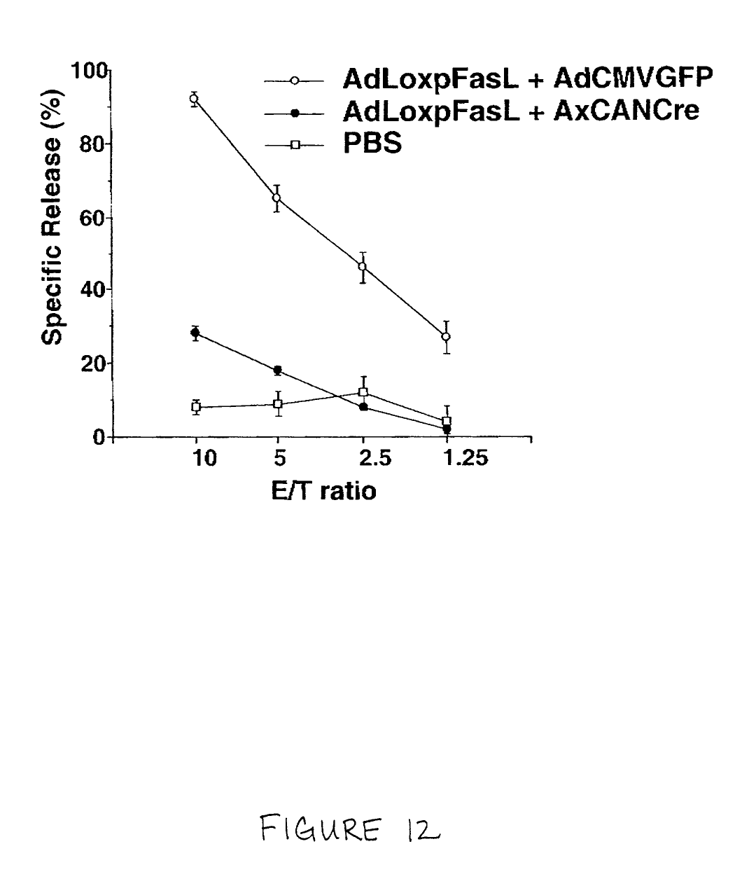

Mice were tolerized in vivo with APC-AdFasL or APC-AdControl and then stimulated in vivo with AdCMVLacZ (10×10¹⁰ pfu i.v.). Seven days later, splenic T-cells were purified and their ability to kill AdCMVGFP-infected antigen presenting cells target cells was determined. After stimulation with AdCMVLacZ, T cells from mice which had been tolerized with APC-AdControl demonstrated high cytotoxic activity against APC infected with AdCMVGFP (FIG. 12). In contrast, mice that had been tolerized with APC-AdFasL and subsequently immunized with AdCMVLacZ exhibited low cytotoxic activity against the AdCMVGFP-infected antigen presenting cells.

### EXAMPLE 23

T-cell Tolerance Demonstrated by Decreased IFN-γ and IL-2 Production

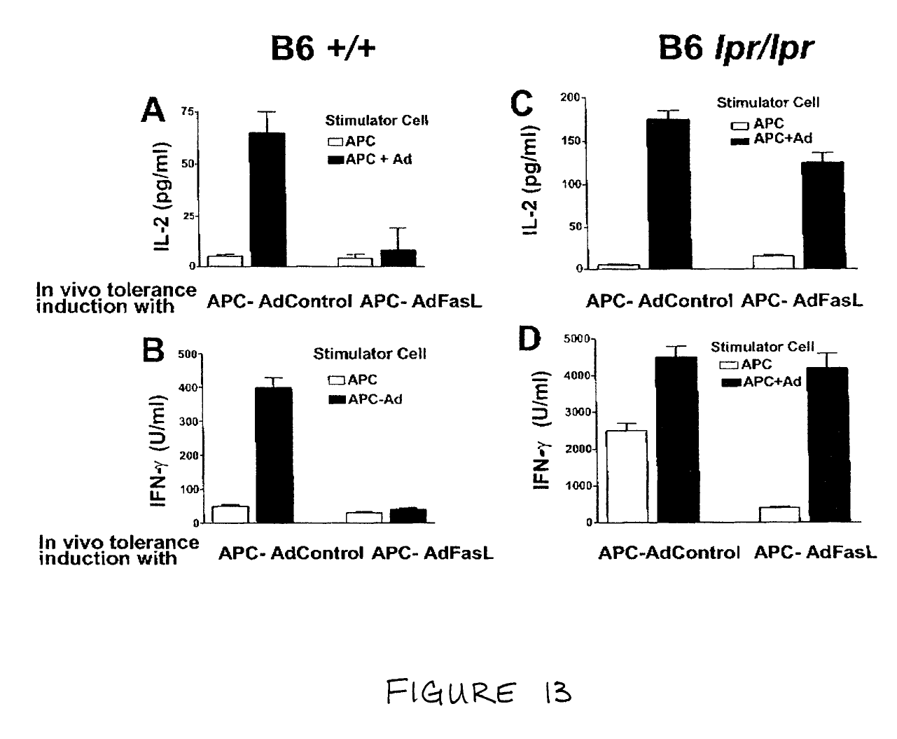

Thirty days after treatment with either APC-AdFasL or APC-AdControl, mice were sacrificed and the spleen cells stimulated with either antigen presenting cells or antigen presenting cells infected with AdCMVlacZ The T cells were not stimulated by the non-infected antigen presenting cells as only low levels of IL-2 and IFN-γ was produced (FIGS. 13A, B). Antigen presenting cells infected with AdCMVlacZ stimulated high levels of IL-2 and IFN-γ by spleen cells from untolerized C57BL/6 mice treated with APC-AdControl. In contrast, antigen presenting cells infected with AdCMVlacZ were unable to stimulate IL-2 and IFN-γ production by splenic T cells from B6+/+mice that had been treated with APC-AdFasL (FIGS. 13A, 13B). Tolerance induction by APC-AdFasL required Fas expression in recipient mice since spleen cells from tolerized B6-lpr/lpr mice challenged with AdCMVLacZ produced high levels of IFN-γ and IL-2 (FIGS. 13C, 13D). These results indicate that APC-AdFasL results in long-term, Fas-mediated systemic tolerization of T cells in vivo and induces non-responsiveness in these T cells upon stimulation with APC-AdCMVLacZ 4 weeks after treatment.

### EXAMPLE 24

APC-AdFasL Induces Specific Tolerance to Adenovirus

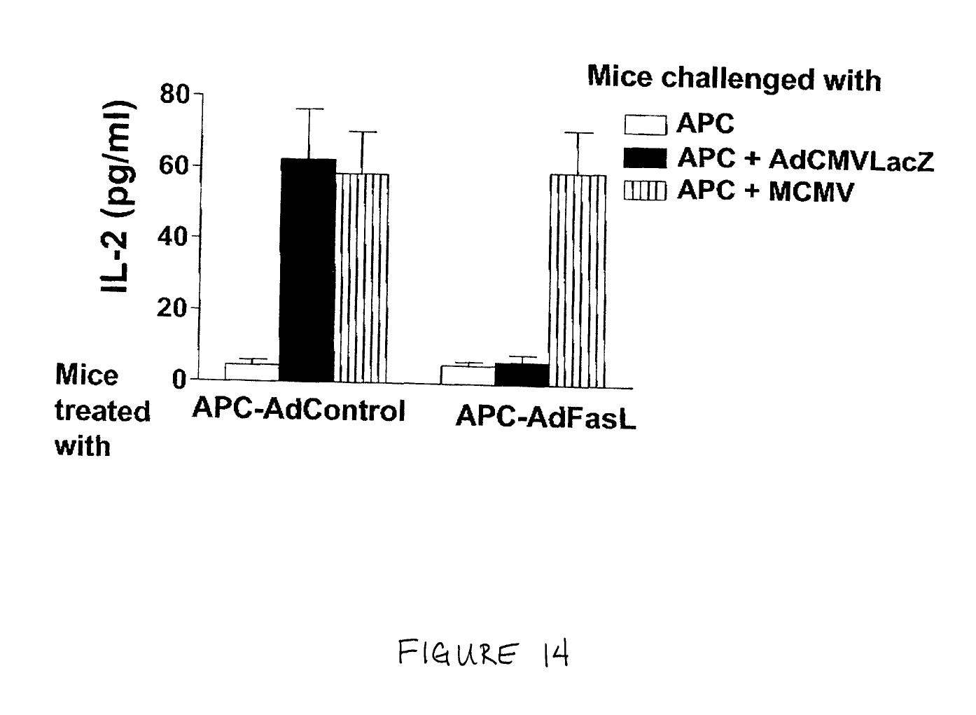

To determine if the T-cell tolerance induced by APC-AdFasL was specific for adenoviral vector rather than general suppression of the immune response to viral infection, the T-cell response of APC-AdFasL and APC-AdControl tolerized mice to murine cytomegalovirus viral (MCMV) infection was evaluated. B6-+/+ mice were treated with APC-AdFasL as described above for induction of tolerance, and then i.v. challenged 7 days later with either adenovirus or MCMV (FIG. 14). After an additional 7 days, splenic T-cells were stimulated in vitro with antigen presenting cells alone, or antigen presenting cells infected with MCMV or AdCMVLacZ. Although there was a remarkable reduction in the T-cell response to adenoviral vector, the T-cell response to MCMV was not impaired as demonstrated by the comparable levels of IL-2 produced by the T cells from both APC-AdControl and APC-AdFasL treated mice. This result indicates that inhibition of the T-cell response in APC-AdFasL tolerized mice is specific for adenoviral vector.

Fas-mediated apoptosis is a critical mechanism in the activation-induced suicide of T cells. In this process, autocrine interaction of Fas and Fas ligand occurs on the same T cell (Ju et al., 1995; Brunner et al., 1995; Dhein et al., 1995). There results presented herein suggest that a paracrine process also plays an important role in Fas-mediated apoptosis in T cells. Apoptosis of T cells mediated by Fas ligand in a paracrine fashion has been shown previously to be critical for the maintenance of the immunoprivileged site. High levels of Fas ligand expression on the surrounding immunoprivileged cells or tissues are able to induce apoptosis in the T cells and prevent T-cell attack. Given the fact that local expression of high levels of Fas ligand can induce an inflammatory response, the role of Fas ligand in induction of local T-cell tolerance has been challenged. A recent study suggests that maintenance of immunoprivilege involves induction of systemic T-cell tolerance (Griffith et al., 1996). Results of the present invention provide direct evidence that Fas ligand-expressing antigen presenting cells induce depletion of responding T cells in the peripheral lymphoid organs and leads to systemic T-cell tolerance to the specific antigen, and suggest a mechanism of immunoprivilege that Fas ligand-bearing cells can be released from immune-privileged tissues leading to systemic T-cell tolerance.

These findings indicate that T-cell apoptosis induced by Fas ligand expressing antigen presenting cells is different from activation-induced T-cell suicide. The former involves direct antigen presentation and occurs early and in a more efficient manner, whereas the latter occurs later after antigen challenge, and the deletion is incomplete. It is not clear whether T-cell apoptosis induced by Fas ligand expressing antigen presenting cells requires that the T cells be activated. However, given the fact that naive T cells can undergo Fas ligand-mediated apoptosis, early activation of T cells may not be required for this form of apoptosis.

Induction of antigen-specific T cell tolerance by Fas ligand expressing antigen presenting cells suggests a novel role of antigen presenting cells in modulation of the T-cell response. Results from the present invention indicate that Fas ligand-expressing antigen presenting cells induce an earlier and more profound clonal deletion of the antigen-reactive T cells than does activation-induced suicide of the T cells. This suggests that T-cell tolerance can be induced by the antigen presenting cells during an early stage of the T-cell response. When naive T cells recognize the antigen presented by antigen presenting cells, the fate of the T cells is determined by the antigen presenting cells: the T cells undergo complete activation if the antigen presenting cells express appropriate co-stimulatory molecules, such as B7, or undergo induction of anergy if the antigen presenting cells do not express co-stimulatory molecules, or undergo apoptosis if the antigen presenting cells express Fas ligand. The ability of antigen presenting cells to present antigen to T cells in either an immunogenic or tolerogenic fashion has been proposed to be a critical mechanism in regulation of the T-cell response during early activation. Activation-induced cell death of lymphocytes mediated by macrophages in an antigen-specific fashion has been proposed as an additional mechanism by which autoreactive T cells are deleted by non-inflammatory tissue macrophages and macrophage-mediated cell deletion plays a role in regulation of B lymphopoiesis. Upregulation of Fas ligand expression in macrophages also has been implicated as a mechanism by which T cells are depleted during HIV infection. Previous studies showed that activated macrophages express Fas ligand. The findings presented herein indicate a novel apoptosis-inducing function of antigen presenting cells, in addition to their known functions in induction of activation and anergy of T cells.

The present invention discloses antigen-specific T-cell tolerance induced by antigen presenting cells that express Fas ligand, and was established in two experimental systems: allogeneic T-cell tolerance and H-Y antigen-specific tolerance in TCR transgenic mice. This suggests that direct antigen presentation by donor antigen presenting cells that express Fas ligand is required for induction of apoptosis in the antigen responding T cells. Direct antigen presentation is a major component of allogeneic T-cell activation and allograft rejection. Antigen presenting cells carrying alloantigens released from the grafted organs and tissues are strongly immunogenic. As they migrate into the peripheral lymphoid organs of the recipients, a strong T-cell response is elicited, and, finally, these activated T-cells attack the grafted tissue resulting in rejection. The present invention suggests a practical immunointervention strategy in induction of systemic and antigen-specific T-cell tolerance by manipulating Fas ligand expression on the antigen presenting cells. This implies that allogeneic T-cell tolerance can be induced and maintained by removal of alloantigen-specific T cells in the recipients using Fas ligand expressing donor antigen presenting cells.

Interaction between T cells and antigen presenting cells plays a major role in initiation of an autoimmune response. Inappropriate antigen presentation by certain MHC molecules has been shown to be a key step toward activation of autoreactive T cells and development of autoimmune disease. The manner in which antigen is presented by antigen presenting cells determines the cytokine profile of the T-cell response, which may be involved in the pathogenesis of autoimmune disease. The rapid elimination of the antigen responding T cells by Fas ligand expressing antigen presenting cells suggests that this strategy would be applicable to the treatment of those autoimmune diseases that are T cell-dependent and of known autoantigen specificity. One can anticipate that the antigen presenting cells carrying autoantigen and Fas ligand may facilitate elimination of the autoantigen-specific T cells by enhancing Fas-mediated apoptosis in the autoreactive T cells.

The present invention further demonstrates extremely efficient inhibition of CD3⁺ T-cell expansion, which are potentially reactive with APC-processed adenovirus antigens, leading to prolongation of gene expression with AdCMVLacZ after tolerance induction by APC-AdFasL. High efficiency inhibition of adenovirus-reactive T-cells was achieved by treatment of mice every 3 days with 5 doses of APC-AdFasL using antigen resenting cells from B6lpr/lpr mice. This protocol led to tolerization to antigens for up to 4 weeks through inhibition of APC/antigen-reactive T-cells. Therefore, administration of AdCMVLacZ (10¹⁰ pfu) intravenously one week after tolerance does not lead to a significant T-cell response since there is deletion or inhibition of all potentially reactive T-cells. One week after challenge with intravenous AdCMVLacZ, there was no visible expansion of CD3⁺ T-cells in the spleen. The absence of cytotoxic T-cells at 7 days post-infection with AdCMVLacZ correlated with a prolonged expression of LacZ in tolerized mice compared to non-tolerized mice. This is the first demonstration that adenovirus expression of Fas ligand within a n APC can be used as pretreatment to tolerize against administration of an adenovirus gene therapy product.

Tolerance induction by antigen presenting cells infected with adenovirus and expressing high levels of Fas ligand is specific for adenovirus, but not MCMV. This is significant since other methods for induction of tolerance to, or immunosuppression of, adenovirus gene therapy are associated with a more generalized immunosuppressed state, which would be undesirable for long-term gene therapy use. However, the present tolerizing technique completely abrogates the ability of the recipient to respond to the tolerizing virus used to infect the APC, but does not affect the response to another virus. Therefore, this method of tolerization prior to adenovirus gene therapy would be widely applicable, would not result in generalizing immune-suppression, and could be re administered for repeated treatment as needed without inducing a n immune-suppressed state.

The following references were cited herein:

Bretscher, P. & Cohn, M. Science 169, 1042-1049 (1970).

Lafferty, J. K. & Gill, R. G. Immuno. Cell. Biol. 71, 209-214 (1993).

Liu, Y. & Linsley, P. S. Curr. Opin. Immunol. 4, 265-270 (1992).

June, C. H., et al. Immunol. Today 15, 321-331 (1994).

Linsley, P. S. & Ledbetter, J. A. Annu. Rev. Immunol. 11, 191212 (1993).

Lenschow, D. J., et al. Annu. Rev. Immunol. 14, 233-258 (1996).

Dhein, J., et al. Nature 373, 438-441 (1995).

Brunner, T., et al. Nature 373, 441-444 (1995).

Ju, S. T., et al. Nature 373, 444-448 (1995).

Itoh, N., et al. Cell 66, 233-243 (1991).

Watanabe-Fukunaga, R., et al. Nature 356, 314-317 (1992).

Suda, T., et al. Cell 75, 1169-1178 (1993).

Takahashi, T., et al. Cell 76, 969-976 (1994).

Bellgrau, D., et al. Nature 377, 630-632 (1995).

Griffith, T. S., et al. Science 270, 1189-1192 (1995).

Griffith, T. S., et al. Immunity 5, 7-16 (1996).

Lau, H. T., et al. Science 273, 109-112 (1996).

Kisielow, P. H., et al. Nature 333, 742-745 (1988).

Zhou, T., et al. J. Immunol. 147, 466-474 (1991).

Finkelman, F. D., et al. J. Immunol. 157, 1406-1414 (1996).

Munn, D. H., et al. J. Immunol. 156, 523-532 (1996).

Osmound, D. G., et al. Immunol. Rev. 142, 209-230 (1994).

Badley, A. D., et al. J. Virol. 70, 199-206 (1996).

Mountz, J. D., et al. J. Immunol. 155, 4829-4837 (1995).

Zhou, T., et al. J. Exp. Med. 183, 1879-1892 (1996).

Yang, Y., and Wilson, J. M. J. Immunol. 155:2564-2570 (1995).

Christ. M., et al. Immunol Letters. 57:19-25 (1997).

Yang, Y., et al. J. Virol. 69:2004-2015 (1995).

Gilgenkrantz, et al. Human Gene Therapy. 6:1265-1274 (1995).

Yang, et al. Proc Natl Acad of Sci USA. 91:4407-4411 (1994).

Juillard, et al. Euro. J. of Immunology. 25:3467-3473 (1995).

Yang, Y., et al. J. of Virology. 70:6370-6377 (1996).

Schowalter, D. B., et al. Gene Therapy. 4:853-860 (1997).

Qin, L., et al. Human Gene Therapy. 8:1365-1374 (1997).

Guerette, B., et al. Human Gene Therapy. 7:1455-1463 (1996).

Zsengeller, Z. K., et al. Human Gene Therapy. 8:935-941 (1997).

French, L. E., et al. J Cell Biol.: 199:335-343 (1996).

Lee,J., et al. Endocrinology 138:2081-2088 (1997).

Suda, T., et al. J Immunol 154:3806-13 (1995).

Zhou, T, et al. J. Exp Med. 176:1063-1072 (1992).

Wu, J., et al. Proc Natl Acad Sci USA. 91:2344-2348 (1994).

Cheng, J.,et al. J. Immunol. 159:674-684 (1997).

Lau, H. T., et al. Science 273:109-112 (1995).

Muruve,D. A., et al. Transplantation. 64:542-546 (1997).

Muruve, D. A., et al. Hum Gene Therapy. 8:955-963 (1997).

Sigalla J. et al. Human Gene Therapy. 8:1625-1634 (1997).

DeMatteo, R. P., et al. Ann Surg 222:229-239 (1995).

Hamilton, T., et al. J. of Pediatric Surgery. 32:373-377 (1997).

Zepeda, M., and Wilson, J. M. Gene Therapy. 3:973-979 (1996).

Ilan, et al. J. of Clinical Investigation. 99:1098-1106 (1997).

Bennett, J. et al. Human Gene Therapy. 7:1763-1769 (1996).

Zhang, H. G., et al. J Virol 72:2483-2490 (1998).

Young, D. C., et al. Anal. Biochem. 215:24-30 (1993).

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of inducing systemic tolerance to a n antigen in an individual in need of such treatment, comprising the step of:
administering antigen presenting cells to said individual, wherein said cells express Fas ligand and said antigen.

2. The method of claim 1, wherein said antigen presenting cells induce apoptosis of Fas-positive T-cells directed towards said antigen, thereby resulting in said induction of specific, systemic tolerance to said antigen.

3. The method of claim 1, wherein said antigen is selected from the group consisting of the adenovirus antigen, a viral antigen, an adeno-associated viral antigen, an autoantigen, and an alloantigen.

4. The method of claim 1, wherein said individual has an autoimmune disease.

5. The method of claim 4, wherein said autoimmune disease is selected from the group consisting of diabetes, multiple sclerosis, rheumatoid arthritis, thyroiditis, Grave's disease, systemic lupus erythematosus.

6. The method of claim 1, wherein said individual has had an organ transplant.

7. The method of claim 1, wherein administration of said antigen presenting cells to said individual results in a decreased cytotoxic T cells and decreased CD4 helper cells.

8. The method of claim 1, further comprising the step of delivering to said antigen presenting cells a gene to inhibit apoptosis.

9. The method of claim 8, wherein said gene to inhibit apoptosis is crmA.

10. A method of inducing T-cell tolerance to a virus in an individual receiving gene therapy, comprising the steps of:
transfecting Fas ligand-expressing antigen presenting cells with said virus;
introducing said transfected antigen presenting cells into said individual; and
treating said individual with said virus for the purpose of gene therapy, wherein said antigen presenting cells expressing the Fas ligand induce T-cell tolerance to said virus.

11. The method of claim 10, further comprising the step of delivering to said antigen presenting cells a gene to inhibit apoptosis.

12. The method of claim 11, wherein said gene to inhibit apoptosis is crmA.

13. A method of increasing expression of a transgene in an individual, comprising the steps of:
administering antigen presenting cells to said individual, wherein said cells express Fas ligand and an antigen to the protein product of said transgene; and
delivering to said individual a viral vector encoding a transgene wherein said antigen presenting cells induce apoptosis of Fas-positive T-cells resulting in an increased expression of the transgene.

14. The method of claim 13, further comprising the step of delivering to said antigen presenting cells a gene to inhibit apoptosis.

15. The method of claim 14, wherein said gene to inhibit apoptosis is crmA.

16. A method of creating immune-privileged sites in an individual so as to decrease rejection of a graft, comprising the steps of:
extracting antigen presenting cells from donor organ tissue;
introducing Fas ligand into said antigen presenting cells to produce Fas ligand-expressing antigen presenting cells expressing an antigen specific to said graft;
introducing said Fas ligand-expressing antigen presenting cells expressing an antigen specific to said graft to said individual prior to and during said grafting procedure; wherein said Fas ligand-expressing antigen presenting cells expressing an antigen specific to said graft create create said immune-privileged site at the site of said grafting procedure to prevent rejection of said graft in said individual.

17. A method decreasing rejection of a graft in an individual, comprising the steps of:
perfusing donor organ tissue with Fas ligand;
introducing said donor organ tissue to said individual.
